(19) United States
(12) Patent Application Publication
(10) Pub. No.: US 2011/0208172 A1
(43) Pub. Date: Aug. 25, 2011

(54) Eye measurement and modeling techniques

(76) Inventors: Gerhard Youssefi, Landshut (DE); Julia Hoff, Muenchen (DE); Birgit Lutzenberger, Hamburg (DE); Anton Hilger, Munchen (DE)

(21) Appl. No.: 13/035,174

(22) Filed: Feb. 25, 2011

Related U.S. Application Data
(63) Continuation of application No. PCT/US2009/054723, filed on Aug. 24, 2009.
(60) Provisional application No. 61/092,487, filed on Aug. 28, 2008.

(51) Int. Cl. A61F 9/01

(52) U.S. Cl. 606/5

(57) ABSTRACT

A refractive surgical system, comprising a refractive treatment apparatus adapted to alter multiple localized regions of a cornea and an ophthalmic measurement device adapted to measure a corneal shape parameter at at least two locations on the cornea. A corneal modeling apparatus and method to calculate anticipated corneal shape parameters at two or more locations based on parameters of a refractive treatment, and compare shape parameters measured at two or more locations on the cornea corresponding to the two or more locations of the anticipated corneal shape parameters.

This is a continuation of International Application PCT/US2009/054723, with an international filing date of Aug. 24, 2009, and which claims the benefit of U.S. Provisional Patent Application No. 61/092,487, with a U.S. filing date of Aug. 28, 2008.

### FIELD OF INVENTION

The present invention relates to apparatus and methods for eye measurement and/or modeling.

### BACKGROUND

It is conventionally known that a low-coherence, time-domain interferometer can be used to measure corneal thickness at a center of a cornea during refractive surgery to monitor a surgical result. It is also known that a measurement output can be compared to an anticipated corneal dimension at the center of the cornea, where the anticipated dimension is calculated based on a model of the surgical procedure. It is further known that such apparatus can be used to provide real-time feedback for controlling a photoablative laser to improve surgical results.

### SUMMARY

The Applicants have recognized that a first limitation of prior art apparatus is their ability to model the cornea at a single location at the center of the cornea.

The Applicants have recognized that another limitation of prior art apparatus is their ability to measure the cornea at a single location at the center of the cornea. For example, limitations of the prior art apparatus are associated with the fact that, to perform certain refractive surgical procedures, a corneal flap is cut into an eye to expose the stromal surface of the cornea. A stromal surface tends to be relatively, highly scattering. Accordingly, after a corneal flap is cut, measurement of the cornea using a conventional low-coherence, time-domain interferometeric measurements is deleteriously affected by to scattering from the stromal surface. A further drawback of such apparatus is that they are capable of measuring thicknesses at only a single corneal location due to a need to collect light that is specularly reflected from surfaces of the eye.

Aspects of the present invention are directed to three-dimensional corneal modeling methods and apparatus. These aspects are useful when used in conjunction with a measurement apparatus capable of making three-dimensional measurements of a cornea (e.g., to form a surgical feedback apparatus) and/or a refractive surgical apparatus.

However, modeling apparatus can be used without such measurement or surgical apparatus.

Additional aspects of the present invention are directed to apparatus suitable for measuring corneal shape parameters corresponding to multiple locations across the cornea of an eye. These aspects are useful when used in conjunction with a modeling apparatus and/or a refractive surgical system. However, measurement apparatus can be used without such modeling or surgical apparatus.

An aspect of the invention is directed to a refractive surgical system, comprising a refractive treatment apparatus adapted to alter multiple regions of a cornea, and an ophthalmic measurement device adapted to measure corneal shape parameters at at least two locations on the cornea affected by the treatment apparatus.

In some embodiments, the measurement device comprises a Fourier domain OCT device. In some embodiments, the treatment apparatus comprises a laser. The laser may comprise one of an excimer laser and a femtosecond laser.

In some embodiments, the system is adapted to modify a fluence of the laser in response to the measured corneal shape parameters. In some embodiments, the measured corneal shape parameters are thicknesses of the cornea. In some embodiments, the corneal shape parameters are corneal positions. In some embodiments, the apparatus is configured such that the at least two locations span at least 2 millimeters.

In some embodiments, the measurement device comprises a moveable time-domain OCT device. In some embodiments, the system further comprises a processor adapted to A) calculate anticipated corneal shape parameters at the two or more locations based on parameters of a refractive treatment, and B) compare the shape parameters measured at the two or more locations to the anticipated corneal shape parameters.

Another aspect of the invention is directed to a corneal modeling apparatus comprising a processor adapted to A) calculate anticipated corneal shape parameters at two or more locations based on parameters of a refractive treatment, and B) compare shape parameters measured at two or more locations on a cornea to the anticipated corneal shape parameters. The two or more locations on the cornea correspond to the two or more locations of the anticipated corneal shape parameters.

In some embodiments, the apparatus further comprises a refractive treatment apparatus adapted to perform the refractive treatment on the cornea. In some embodiments, the apparatus further comprises an ophthalmic measurement device adapted to obtain the two or more measured shape parameters.

In some embodiments, the measurement device comprises a Fourier domain OCT device. In some embodiments, the treatment apparatus comprises a laser. In some embodiments, the laser comprises an excimer laser and a femtosecond laser. In some embodiments, the system is adapted to modify a fluence of the laser in response to a difference between the measured shape parameters measured and the anticipated corneal shape parameters. In some embodiments, the system is adapted to notify an operator of the system if a difference between the measured shape parameters measured and the anticipated corneal shape parameters is too great.

In some embodiments, the corneal shape parameters are thicknesses of the cornea. In some embodiments, the corneal shape parameters are corneal positions. The at least two measurement locations may span at least 2 millimeters.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic block diagram of an example of a refractive surgical apparatus according to aspects of the present invention;

FIG. 2 is a schematic illustration of an example of an embodiment of a refractive surgical apparatus according to aspects of the present invention;

FIG. 3 is a schematic illustration of another embodiment of a refractive surgical apparatus according to aspects of the present invention; and

FIG. 4 is a flowchart illustrating one example of a technique according to aspects of the present invention.

### DETAILED DESCRIPTION

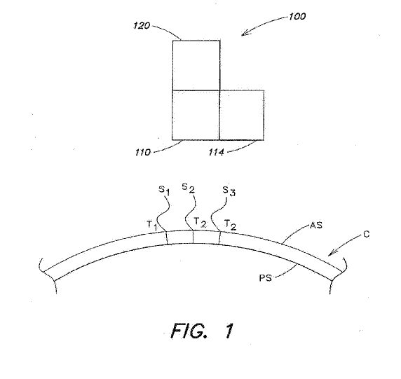

FIG. 1 is a schematic block diagram of an example of a refractive surgical apparatus 100 according to aspects of the present invention adapted to project light onto a cornea C. Apparatus 100 comprises an interferometer 110, a treatment laser 120, and a processor 114. The processor is configured as a three-dimensional corneal modeling apparatus. The processor is programmed 1) to calculate anticipated corneal shape parameters at two or more locations across a cornea of an eye, at one or more times during a refractive surgical procedure, as described in greater detail below with reference to processor 214, and 2) to compare an anticipated corneal shape parameter to a measured corneal shape parameter at the two or more locations across a cornea of an eye, at the one or more times during a refractive surgical procedure.

Interferometer 110 is configured to be capable of measuring corneal shape at multiple locations S₁, S₂ S₃ along a cornea C. Although measurements at three locations are shown, apparatus according to aspects of the present invention are configured to measure a corneal shape parameter at two or more locations along the cornea. In some embodiments, the number of locations in the corneal region to be measured is in the range of hundreds up to thousands. Typically, the corneal region in which measurements are made is a circular region having a diameter of 6-8 mm; however, a region of any suitable size and shape may be used.

Treatment laser 120 may be any suitable treatment laser (e.g., eximer laser or a femtosecond laser). Laser 120 is typically configured in a manner to permit control of a fluence output of the laser. For example, a laser may be so configured by controlling a current or voltage input to the laser. Alternatively, the laser may be provided with a controllable optical filter having a variable transmission. Although a laser is shown, any other treatment apparatus capable of separately treating multiple locations of a cornea may be used.

As described in greater detail below, interferometer 110 provides measurements at the multiple locations and determines whether a particular corneal shape or shape change has been achieved at a particular point in time during the treatment. In the event that the particular shape is not achieved, notification (e.g., visual, audio, tactile notification) is provided to an operator and/or laser 120 is controlled to achieve a particular shape. The laser control may include one or more of an increase or decrease in fluence, or an alteration (i.e., an increase or decrease) in the number of photoablative shots output from the laser, or an alteration of the location of photoablative shots output from the laser.

A shape parameter of the cornea can be determined using at least one of the following two techniques, although other techniques may be used. In a first technique, pachymetry (i.e., thickness) of the cornea at the multiple locations is determined. According to this technique, the interferometer output is used to determine the distance between the anterior surface AS of the cornea and the posterior surface PS of the cornea at the locations. In a second technique, only positions of the cornea (e.g. positions of the anterior surface of the cornea) at the multiple locations are determined. It will be appreciated that if only the anterior surface is measured, it is typically desirable that care be taken to maintain a known distance between the cornea and a reference location (e.g., a surface of the interferometer measurement apparatus).

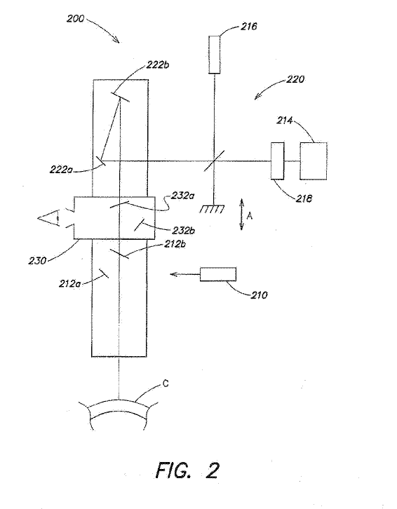

FIG. 2 is a schematic illustration of an example of an embodiment 200 of a refractive surgical apparatus according to aspects of the present invention comprising a treatment laser system 210, a Fourier domain optical coherence tomography (OCT) device 220, and a processor 214.

Laser system 210 is configured to perform a course of refractive treatment. Although a laser system is illustrated, any other apparatus capable of a performing a refractive treatment may be used. The term “refractive treatment” as used herein refers to ablation or corneal structural-changing treatments whether achieved by a laser or other apparatus capable of altering multiple regions of a cornea. It will be appreciated that such treatments achieve a change in refraction of an eye.

Laser system 210 may comprise any laser suitable for performing a controllable treatment of a cornea. For example, a laser capable of providing a controllable treatment may be configured to provide a variable output fluence and/or variable laser shot locations. In the illustrated apparatus, shot locations are determined by suitably positioning steering mirrors 212a, 212b.

Fourier domain OCT device 220 device is adapted to measure a corneal shape parameter at at least two locations on the cornea. Such measurements may be made a locations areas affected by system 210. Location at which measurements are made is determined by suitably positioning steering mirrors 222a, 222b. Although system 210 ss shown as comprising a single apparatus capable of altering multiple regions of a cornea (i.e., a single laser), multiple such apparatus may be used.

OCT device 220 comprises a light source 216 having a suitably short temporal coherence to permit low coherence interferometery measurements to be performed at the multiple locations, and processor 214 is programmed to determine the location of the front surface and/or rear surface of the cornea. It will be appreciated that processor 214 is adapted to perform appropriate calculations (e.g., including Fourier transforming) to determine a measured corneal shape. The processor may also be programmed to calculate a shape of the anterior surface of the cornea and/or the shape of posterior surface of the cornea.

Apparatus according aspects of the present invention are capable of measuring the eye at multiple positions on a corneal surface. The Applicant's have determined that Fourier domain OCT (also commonly referred to as spectral domain) apparatus are particularly appropriate for measuring at multiple locations across a cornea after a corneal flap is cut, due to the fact that they are capable of receiving light from the cornea that is suitable to achieve a measurement signal having an adequate signal-to-noise ratio even when the light is scattered (i.e., the light is not specularly reflected) from a surface of the eye (e.g., an exposed stromal layer of the eye). It will be appreciated that the number of points at which a corneal measurement is made is dependent on the purpose of the measurement.

In some embodiments, a surgical microscope may be provided to permit an operator to view the cornea via steering mirrors 232a, 232b.

Device 220 may be configured to employ any suitable Fourier domain OCT technique. For example, device 220 may be a spectral domain, Fourier OCT comprising a grating (not shown) to spatially disperse the spectrum across an array-type detector (e.g., detector 218). Alternatively, device 220 may comprise a swept source (SS) Fourier OCT using a narrow band laser (not shown) capable of outputting a light of variable wavelength, thereby encoding the spectrum as a function of time.

One appropriate technique for specifying the shapes of the surfaces of the corneal surfaces is expressed by using the corneal surface data to calculate the magnitudes of Zernike polynomials. It will be appreciated that, if two or three positions on a corneal surface are measured, only second-order Zernike polynomial coefficients can be accurately calculated. That is, the spherical shape and the cylindrical shape can be determined. If ten points on a corneal surface are measured, then third order Zernike polynomials coefficients can be calculated. If fifteen points on a corneal surface are measured, then fourth order Zernike polynomials coefficients can be calculated. That is, defocus, spherical aberration, second order astigmatism, coma, trefoil can be calculated. In some embodiments, the corneal measurement results are used to calculate Zernike polynomials corresponding to a corneal surface as described above; however, any suitable surface characterizing data may be extracted from the measurement data.

It will be appreciated that the above-specified numbers of points represent the approximate minimum number of points to be used for each calculation and, by increasing the number of points for a given calculation, the stability of the calculation can be enhanced. In some embodiments, at least one hundred points are calculated and in other embodiments at least one thousand points are calculated. To achieve a large number of points, steering mirrors 222a and 222b can be suitably positioned to project light onto the eye and to receive scattered light from source 216 after it is scattered from the eye.

In the illustrated embodiment, processor 214 is configured to 1) calculate anticipated, corneal shape parameters at two or more locations across a cornea of an eye, at one or more times during a refractive surgical procedure, and 2) compare an anticipated corneal shape parameter to a measured corneal shape parameter at two or more locations across a cornea of an eye, at one or more times during a refractive surgical procedure. Further details regarding the calculation and comparison are given below. In some embodiments, surface calculations are made during breaks in the laser treatment. In some embodiments, the time needed to measure and calculate values is less than 0.5 seconds to keep the time low and thereby reduce eye movement that occurs during measurement and calculation. It will be appreciated that, although in the illustrated embodiment, a single processor is shown for calculating, measuring and comparing parameters, two or more processor may be used to accomplish these tasks.

In some embodiments, the apparatus 200 is configured to make measurements over a 6 mm diameter circular area corresponding to a dilated pupil diameter. In other embodiments, the measurement area spans at least 2 mm or at least 3 mm. Steering mirrors 222a, 222b are moveable to appropriately direct light to cornea C and from cornea C.

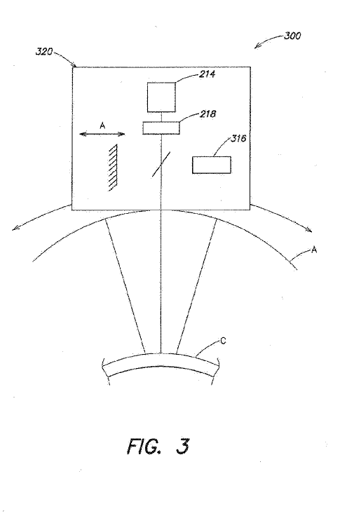

FIG. 3 is a schematic illustration of another embodiment of a refractive surgical apparatus 300 according to aspects of the present invention comprising a treatment laser system (not shown) and a moveable time-domain OCT apparatus. Housing 320 is moveable along an arc such that light from source 316 can be specularly reflected from cornea C and received by detector 318 at two or more locations on the cornea. In some embodiments, the apparatus is moveable such that measurements can be made over a 6 mm diameter circular area. It will be appreciated that although the arc A is illustrated in two dimensions, it will typically extend in three-dimensions (e.g., spherical, ovoid or other possibly more complicated shapes).

A disadvantage of an apparatus 300 is the need to receive specularly reflected light from the cornea. However, it will be appreciated that, to achieve such a result, an anterior surface of the cornea can be determined prior to surgery (e.g., using data from a slit scan pachymeter, or a Placido topographer) or the interferometer can be appropriately tilted for each measurement location to achieve a suitable signal-to-noise in the output signal of the interferometer.

An aspect of the invention is directed to techniques for modeling a cornea (e.g., using processor 214) in conjunction with or apart from measurement. A corneal biological modeling apparatus according to aspects of the present invention comprises a processor programmed to calculate, anticipated corneal shape parameters at two or more locations across a cornea of an eye. The processor is adapted to calculate the shape parameters expected to occur at a given time. Additional anticipated shape parameters may be calculated for one or more additional times.

In some embodiments, the processor is also programmed to compare the anticipated corneal shape parameters to measured corneal shape parameters at the two or more locations across a cornea of an eye, at the one or more times during a refractive surgical procedure. This aspect of the invention may be used, for example, with a refractive treatment apparatus as described above, where the measured shape may, for example, be an input from OCT device 220. For example, the techniques may be used to control a treatment laser. It will be appreciated that the term “corneal shape” refers to a three-dimensional configuration of the cornea, and that the term “shape parameter” refers to a thickness or other dimensional parameter. Such a parameter can be measured at x, y locations across a corneal surface, thereby providing three-dimensional corneal information.

For example, in embodiments where the techniques are used to control a laser, the result of the surface measurement may be compared with the calculated, anticipated corneal shape or shape parameters as was discussed above. In embodiments where a measurement result is compared to an anticipated shape, the laser fluence and/or laser shot pattern may be modified, a warning message may be presented to the operator, or a surgery may be terminated if the measured shape deviates from the calculated shape by more than a predetermined amount.

According to one technique, an anticipated corneal shape or shape change is calculated by determining a relationship between corneal shape parameters, and various parameters associated with a refractive treatment. The relationship may be determined as a function of x-y and time t. Equation 1 illustrates one example of an equation suitable for expressing the relationship between various parameters and a resulting pachymetric shape P(x,y).

P(x,y)−P1(x,y)t+P2(x,y)t²+P3(x,y)*V(x,y,t)+P4(x,y)*S(x,y,t)+P5; (1)

where P1 and P2 are spatially-variable coefficients indicating how pachymetry changes proportional to time and proportional to time-squared, respectively (e.g., said terms may model dehydration of coreal tissue as a function of time);

P3 is a spatially-variable coefficients indicating how pachymetry changes proportional to total tissue removal V up to a time t (e.g., the term is dependent on a treatment laser shot pattern as a function of x,y);

P4 is a spatially-variable coefficient indicating how pachymetry changes proportional to tissue removal S at a specific location x,y at a time t; and

P5 is a constant value to offset or to compensate for pre-ablative measurement error.

To populate the model expressed in Equation 1, the values of the coefficients as a function of spatial location x,y and time can be calculated using a regression technique (e.g., using singular value decomposition), by measuring the corneas of multiple patients to determine a relationship between cornea shape parameters and refractive treatment parameters. For example, shape parameters may be measured after a known time, and after a known number of laser pulses have been applied at known locations on the patients' corneas. In some embodiments, coefficients may be further characterized to permit calculated anticipated corneal shape to depend on humidity and temperature conditions under which a surgery occurs. In some embodiments, coefficients may be further characterized to permit anticipated corneal shape to depend on the thickness of flap cut in the eye and/or the type of flap cut (e.g., PRK or Lasik). Additional treatment parameters that could be modeled include, the laser beam profile (e.g., flat top or Gaussian), the application of irrigation or pharmaceuticals, the age of a patient, or the geometry of the patient's cornea. It will be appreciated that a processor can be programmed to populate a model as described herein and/or calculate an anticipated shape or shape change based on parameters of the refractive treatment that is performed.

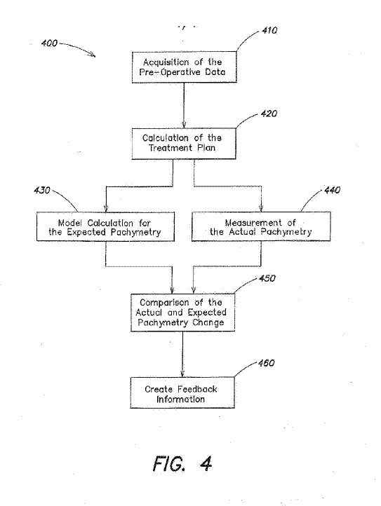

FIG. 4 is a flowchart illustrating one example of a technique 400 according to aspects of the present invention. At step 410, preoperative data is collected regarding a patient's eye. The data would may include corneal shape parameters 1) to provide a starting point of a course of treatment, 2) to calculate a course of treatment, and/or 3) as an input to a model.

At step 420, a course of treatment (including appropriate treatment parameters) is determined using any suitable technique,

At step 430, a model of the anticipated shape is calculated, for example, using parameters of the calculated course of treatment and preoperative measurements as inputs into a model, such as a model of the form of Equation 1. During some courses of treatment, the laser pulses may be applied during two or more phases. For example, if the spherical power of the eye is to be changed by 6.0 diopters, the procedure may occur during 4 phases, during each phase pulses being applied to the eye as appropriate to achieve a change of 1.5 diopters. In such instances, it may be appropriate to measure an eye during a time interval between the phases; however, measurement may be made more or less frequently, including during treatment.

At step 440, a measurement apparatus, for example, as described above with one of FIGS. 1 and 2 is used to measure the actual corneal parameters.

At step 450, a comparison of the anticipated shape and the actual shape is done. If the difference is greater than a selected threshold, then appropriate action is taken, as set forth below. A comparison may be performed at one or more specific locations on the cornea or may be performed using a global shape comparison, such as an RMS calculation.

At step 460, any appropriate action occurs, for example, one or more of: alerting of surgical personal; modification of a shot pattern: termination of a treatment to avoid damaging a subject's eye; or a change in fluence of the treatment laser.

It will be appreciated that, if a patient's cornea is thicker than anticipated at all locations, it can be taken as an indication that the fluence of the laser should be increased; and, if a patient's cornea is thinner than anticipated at all locations, it can be taken as an indication that the fluence should be decreased.

If the patient's cornea assymetrically varies from the anticipated shape (e.g., due to some inhomogeneity of a patient's tissue), then a shot pattern may be altered to achieve an appropriate shape.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. A refractive surgical system, comprising:
a refractive treatment apparatus adapted to alter multiple regions of a cornea; and
an ophthalmic measurement device adapted to measure corneal shape parameters at at least two locations on the cornea affected by the treatment apparatus.

2. The system of claim 1, wherein the measurement device comprises a Fourier domain OCT device.

3. The system of claim 1, wherein the treatment apparatus comprises a laser.

4. The system of claim 3, wherein the laser comprises one of an excimer laser and a femtosecond laser.

5. The system of claim 3, wherein the system is adapted to modify a fluence of the laser in response to the measured corneal shape parameters.

6. The system of claim I, wherein the corneal shape parameters are thicknesses of the cornea.

7. The system of claim 1, wherein the corneal shape parameters are corneal positions.

8. The system of claim 1, wherein the at least two locations span at least 2 millimeters.

9. The system of claim 1, wherein the measurement device comprises a moveable time-domain OCT device.

10. The system of claim 1, further comprising a processor adapted to A) calculate anticipated corneal shape parameters at the two or more locations based on parameters of a refractive treatment, and B) compare the shape parameters measured at the two or more locations to the anticipated corneal shape parameters.

11. The apparatus of claim 10, wherein the system is adapted to modify a fluence of the laser in response to a difference between the measured shape parameters measured and the anticipated corneal shape parameters.

12. The apparatus of claim 11, wherein the system is adapted to notify an operator of the system if a difference between the measured shape parameters measured and the anticipated corneal shape parameters is too great.

13. A corneal modeling apparatus comprising a processor adapted to A) calculate anticipated corneal shape parameters at two or more locations based on parameters of a refractive treatment, and B) compare shape parameters measured at two or more locations on a cornea to the anticipated corneal shape parameters, the two or more locations on the cornea corresponding to the two or more locations of the anticipated corneal shape parameters.

14. The apparatus of claim 13, further comprising a refractive treatment apparatus adapted to perform the refractive treatment on the cornea.

15. The apparatus of claim 14, further comprising an ophthalmic measurement device adapted to obtain the two or more measured shape parameters.

16. The apparatus of claim 15, wherein the measurement device comprises a Fourier domain OCT device.

17. The apparatus of claim 14, wherein the treatment apparatus comprises a laser.

18. The apparatus of claim 17, wherein the laser comprises an excimer laser and a femtosecond laser.

19. The apparatus of claim 17, wherein the system is adapted to modify a fluence of the laser in response to a difference between the measured shape parameters measured and the anticipated corneal shape parameters.

20. The apparatus of claim 19, wherein the system is adapted to notify an operator of the system if a difference between the measured shape parameters measured and the anticipated corneal shape parameters is too great.

21. The apparatus of claim 13, wherein the corneal shape parameters are thicknesses of the cornea.

22. The apparatus of claim 13, wherein the corneal shape parameters are corneal positions.

23. The apparatus of claim 13, wherein the at least two locations span at least 2 millimeters.

24. A corneal modeling method comprising:
calculating anticipated corneal shape parameters at two or more locations based on parameters of a refractive treatment; and
comparing shape parameters measured at two or more locations on a cornea to the anticipated corneal shape parameters, the two or more locations on the cornea corresponding to the two or more locations of the anticipated corneal shape parameters.

25. The method of claim 24, further comprising a refractive treatment apparatus adapted to perform the refractive treatment on the cornea.

26. The method of claim 25, further comprising an ophthalmic measurement device adapted to obtain the two or more measured shape parameters.

27. The method of claim 26, wherein the measurement device comprises a Fourier domain OCT device.

28. The method of claim 25, wherein the treatment apparatus comprises a laser.

29. The method of claim 28, wherein the laser comprises an excimer laser and a femtosecond laser.

30. The method of claim 28, wherein the system is adapted to modify a fluence of the laser in response to a difference between the measured shape parameters measured and the anticipated corneal shape parameters.

31. The method of claim 30, wherein the system is adapted to notify an operator of the system if a difference between the measured shape parameters measured and the anticipated corneal shape parameters is too great.

32. The method of claim 24, wherein the corneal shape parameters are thicknesses of the cornea.

33. The method of claim 24, wherein the corneal shape parameters are corneal positions.

34. The method of claim 24, wherein the at least two locations span at least 2 millimeters.

35. One or more processor readable storage devices having processor readable code embodied on said processor storage devices, said processor readable code for programming one or more processors to execute a method for providing a corneal modeling, comprising:
calculating anticipated corneal shape parameters at two or more locations based on parameters of a refractive treatment; and
comparing shape parameters measured at two or more locations on a cornea to the anticipated corneal shape parameters, the two or more locations on the cornea corresponding to the two or more locations of the anticipated corneal shape parameters.

36. The processor readable storage device of claim 35, further comprising a refractive treatment apparatus adapted to perform the refractive treatment on the cornea.

37. The processor readable storage device of claim 36, further comprising an ophthalmic measurement device adapted to obtain the two or more measured shape parameters.

38. The processor readable storage device of claim 37, wherein the measurement device comprises a Fourier domain OCT device.

39. The processor readable storage device of claim 36, wherein the treatment apparatus comprises a laser.

40. The processor readable storage device of claim 39, wherein the laser comprises an excimer laser and a femtosecond laser.

41. The processor readable storage device of claim 39, wherein the system is adapted to modify a fluence of the laser in response to a difference between the measured shape parameters measured and the anticipated corneal shape parameters.

42. The processor readable storage device of claim 40, wherein the system is adapted to notify an operator of the system if a difference between the measured shape parameters measured and the anticipated corneal shape parameters is too great.

43. The processor readable storage device of claim 35, wherein the corneal shape parameters are thicknesses of the cornea.

44. The processor readable storage device of claim 35, wherein the corneal shape parameters are corneal positions.

45. The processor readable storage device of claim 35, wherein the at least two locations span at least 2 millimeters.
